(19) United States
(12) Patent Application Publication
(10) Pub. No.: US 2004/0225210 A1
(43) Pub. Date: Nov. 11, 2004

(54) Electrode lead-set for use with bioelectric signal detection/acquisition devices

(76) Inventors: John A. Brosovich, Pittsburgh, PA (US); Michael J. Yanniello, Cheswick, PA (US); Albert J. Yarzebinski, Glenshaw, PA (US)

Correspondence Address: GREGORY L BRADLEY, MEDRAD INC, ONE MEDRAD DRIVE, INDIANOLA, PA, 15051

(21) Appl. No.: 10/770,774

(22) Filed: Feb. 2, 2004

Related U.S. Application Data
(60) Provisional application No. 60/444,075, filed on Jan. 31, 2003.

(51) Int. Cl. A61B 5/04

(52) U.S. Cl. 600/372; 600/509

(57) ABSTRACT

A lead set assembly is capable of coupling electrodes to a separate device. The assembly includes two leads. The condition sense lead features two conductive paths, each being connectible to one electrode for conveying a bioelectric signal therefrom and capable of having a first noise signal induced therein by electromagnetic emanations. The noise pickup lead also features two conductive paths, each being effectively topographically aligned with one conductive path of the condition sense lead to form a noise-matched pair therewith and capable of having a second noise signal induced therein by the electromagnetic emanations. In each noise-matched pair, this enables the second noise signal to be substantially approximate to the first noise signal. The lead set assembly can thus be used to convey to the separate device (I) the bioelectric and first noise signals using the condition sense lead and (II) the second noise signals using the noise pickup lead.

### FIELD OF THE INVENTION

The invention generally relates to lead sets of the type used to conduct electrical currents from the surface of living tissues to some device that may use those signals for diagnostic or control purposes. More particularly, the invention relates to systems and methods, and associated lead set assemblies therefor, for conveying electrophysiological signals from a patient located in the bore of a magnetic resonance (MR) scanner, with the signals accompanied by as little noise as possible. Even more particularly, the invention pertains to a lead set assembly for coupling a separate device that uses such bioelectric signals to a patient from which such signals can be detected by use of a plurality of electrodes.

### BACKGROUND OF THE INVENTION

The following information is provided to assist the reader to understand the various environments in which the invention disclosed herein will typically be used.

Magnetic resonance imaging (MRI) is a noninvasive method of producing high quality images of the interior of the human body. It allows medical personnel to see inside the body (e.g., organs, muscles, nerves, bones, and other structures) without surgery or the use of potentially harmful ionizing radiation such as X-rays. The images are of such high resolution that disease and other pathological conditions can often be visually distinguished from healthy tissue. Magnetic resonance (MR) systems and techniques have also been developed for performing spectroscopic analyses by which the chemical content of tissue or other material can be ascertained.

MR imaging and spectroscopic procedures are performed in what is known as an MR suite. As shown in FIG. 1A, an MR suite typically has three rooms: a scanner room 1, a control room 2, and an equipment room 3. The scanner room 1 houses the MR scanner 10 into which a patient is moved via a slideable table 11 to undergo a scanning procedure, and the control room 2 contains a computer console 20 from which the operator controls the overall operation of the MR system. In addition to a door 4, a window 5 is typically set in the wall separating the scanner and control rooms to allow the operator to observe the patient during such procedures. The equipment room 3 contains the various subsystems necessary to operate the MR system. The equipment includes a power gradient controller 31, a radio frequency (RF) assembly 32, a spectrometer 33, and a cooling subsystem 34 with which to avoid the build up of heat which, if left unaddressed, could otherwise interfere with the overall performance of the MR system. These subsystems are typically housed in separate cabinets, and are supplied electricity through a power distribution panel 12 as are the scanner 10 and the slideable patient table 11.

An MR system obtains such detailed images and spectroscopic results by taking advantage of a basic property of the hydrogen atom, which is found in abundance in all cells within the body. Within the body's cells, the nuclei of hydrogen atoms naturally spin like a top, or precess, randomly in every direction. When subject to a strong magnetic field, however, the spin-axes of the hydrogen nuclei align typically themselves in the direction of that field. This is because the nucleus of the hydrogen atom has what is referred to as a large magnetic moment, which is basically an inherent tendency to line up with the direction of the magnetic field to which it is exposed. During an MR scan, the entire body or even just one region thereof is exposed to such a magnetic field. This causes the hydrogen nuclei of the exposed region(s) to line up—and collectively form an average vector of magnetization—in the direction of that magnetic field.

As shown in FIGS. 1B and 1C, the scanner 10 is comprised of a main magnet 101, three gradient coils 103a-c, and, usually, an RF antenna 104 (often referred to as the whole body coil). Superconducting in nature, the main magnet 101 is typically cylindrical in shape. Within its cylindrical bore, the main magnet 101 generates a strong magnetic field, often referred to as the B₀ or main magnetic field, which is both uniform and static (non-varying). For a scanning procedure to be performed, the patient must be moved into this cylindrical bore, typically while supine on table 11, as best shown in FIGS. 1B and 1C. The main magnetic field is oriented along the longitudinal axis of the bore, referred to as the z direction, which compels the magnetization vectors of the hydrogen nuclei in the body to align themselves in that direction. In this alignment, the hydrogen nuclei are prepared to receive RF energy of the appropriate frequency from RF coil 104. This frequency is known as the Larmor frequency and is governed by the equation ω=γ B₀, where ω is the Larmor frequency (at which the hydrogen atoms precess), γ is the gyromagnetic constant, and B₀ is the strength of the main magnetic field.

The RF coil 104 is typically used both to transmit pulses of RF energy and to receive the resulting magnetic resonance (MR) signals induced thereby in the hydrogen nuclei. Specifically, during its transmit cycle, the coil 104 broadcasts RF energy into the cylindrical bore. This RF energy creates a radio frequency magnetic field, also known as the RF B₁ field, whose magnetic field lines point in a direction perpendicular to the magnetization vectors of the hydrogen nuclei. The RF pulse (or B₁ field) causes the spin-axes of the hydrogen nuclei to tilt with respect to the main (B₀) magnetic field, thus causing the net magnetization vectors to deviate from the z direction by a certain angle. The RF pulse, however, will affect only those hydrogen nuclei that are precessing about their axes at the frequency of the RF pulse. In other words, only the nuclei that “resonate” at that frequency will be affected, and such resonance is achieved in conjunction with the operation of the three gradient coils 103a-c.

Each of the three gradient coils is used to vary the main (B₀) magnetic field linearly along only one of the three spatial directions (x,y,z) within the cylindrical bore. Positioned inside the main magnet as shown in FIG. 1C, the gradient coils 103a-c are able to alter the main magnetic field on a very local level when they are turned on and off very rapidly in a specific manner. Thus, in conjunction with the main magnet 101, the gradient coils can be operated according to various imaging techniques so that the hydrogen nuclei—at any given point or in any given strip, slice or unit of volume—will be able to achieve resonance when an RF pulse of the appropriate frequency is applied. In response to the RF pulse, the precessing hydrogen nuclei in the selected region absorb the RF energy being transmitted from RF coil 104, thus forcing the magnetization vectors thereof to tilt away from the direction of the main (B₀) magnetic field. When the RF coil 104 is turned off, the hydrogen nuclei begin to release the RF energy they just absorbed in the form of magnetic resonance (MR) signals, as explained further below.

One well known technique that can be used to obtain images is referred to as the spin echo imaging technique. Operating according to this MR sequence, the MR system first activates one gradient coil 103a to set up a magnetic field gradient along the z-axis. This is called the “slice select gradient,” and it is set up when the RF pulse is applied and is shut off when the RF pulse is turned off. It allows resonance to occur only within those hydrogen nuclei located within a slice of the region being imaged. No resonance will occur in any tissue located on either side of the plane of interest. Immediately after the RF pulse ceases, all of the nuclei in the activated slice are “in phase,” i.e., their magnetization vectors all point in the same direction. Left to their own devices, the net magnetization vectors of all the hydrogen nuclei in the slice would relax, thus realigning with the z direction. Instead, however, the second gradient coil 103b is briefly activated to create a magnetic field gradient along the y-axis. This is called the “phase encoding gradient.” It causes the magnetization vectors of the nuclei within the slice to point, as one moves between the weakest and strongest ends of this gradient, in increasingly different directions. Next, after the RF pulse, slice select gradient and phase encoding gradient have been turned off, the third gradient coil 103c is briefly activated to create a gradient along the x-axis. This is called the “frequency encoding gradient” or “read out gradient,” as it is only applied when the MR signal is ultimately measured. It causes the relaxing magnetization vectors to be differentially re-excited, so that the nuclei near the low end of that gradient begin to precess at a faster rate, and those at the high end pick up even more speed. When these nuclei relax again, the fastest ones (those which were at the high end of the gradient) will emit the highest frequency of radio waves and the slowest ones emit the lowest frequencies.

The gradient coils 103a-c therefore spatially encode these radio waves, so that each portion of the region being imaged is uniquely defined by the frequency and phase of its resonance signal. In particular, as the hydrogen nuclei relax, each becomes a miniature radio transmitter, giving out a characteristic pulse that changes over time, depending on the local microenvironment in which it resides. For example, hydrogen nuclei in fats have a different microenvironment than do those in water, and thus emit different pulses. Due to these differences, in conjunction with the different water-to-fat ratios of different tissues, different tissues emit radio signals of different frequencies. During its receive cycle, RF coil 104 detects these miniature radio emissions, which are often collectively referred to as the MR signal(s). From the RF coil 104, these unique resonance signals are conveyed to the receivers of the MR system where they are converted into mathematical data. The entire procedure must be repeated multiple times to form an image with a good signal-to-noise ratio (SNR). Using multidimensional Fourier transformations, the MR system then converts the mathematical data into a two- or even a three-dimensional image of the body, or region thereof, that was scanned.

When more detailed images of a specific part of the body are needed, a local coil is often used in addition to, or instead of, the whole body coil 104. A local coil can take the form of a volume coil or a surface coil. A volume coil is used to surround or enclose a volume (e.g., a head, an arm, a wrist, a knee or other region) to be imaged. Some volume coils (e.g., for imaging the head and/or extremities) are often referred to as birdcage coils due to their shape. A surface coil, however, is merely fitted or otherwise placed against a surface (e.g., a shoulder, a breast, etc.) of the patient so that the underlying region can be imaged. A local coil can also be designed to operate either as a receive-only coil or a transmit/receive (T/R) coil. A receive-only coil is only capable of detecting the MR signals produced by the body (in response to the RF B₁ magnetic field generated by the body coil 104 during a scanning procedure). A T/R coil, however, is capable of both receiving the MR signals as well as transmitting the RF pulses that produce the RF B₁ magnetic field, which is the prerequisite for inducing resonance in the tissues of the region of interest.

As shown partially in FIGS. 1A and 1C, the scanner room 1 is shielded to prevent the entry and exit of electromagnetic waves. Specifically, the materials and design of its ceiling, floor, walls, door, and window effectively form a barrier or shield 6 that prevents the electromagnetic signals generated during a scanning procedure (e.g., the RF energy) from leaking out of scanner room 1. Likewise, shield 6 is designed to prevent external electromagnetic noise from leaking into the scanner room 1. The shield 6 is typically composed of a copper sheet material or some other suitable conductive layer. The window 5, however, is typically formed by sandwiching a wire mesh material between sheets of glass or by coating the window with a thin layer of conductive material to maintain the continuity of the shield. The conductive layer also extends to the door 4, which when open allows access to the scanner room 1 and yet when closed is grounded to and constitutes a part of shield 6. The ceiling, floor, door and walls of shield 6 provide approximately 100 decibels (dB) of attenuation, and window 5 approximately 80 dB, for the typical operating range of MR scanners (˜20 to 200 MHz). Barrier 6 thus shields the critical components (e.g., scanner, preamplifiers, receivers, local coils, etc.) of the MR system from undesirable sources of electromagnetic radiation (e.g., radio signals, television signals, and other electromagnetic noise present in the local environment).

The shield 6 serves to prevent external electromagnetic noise from interfering with the operation of the scanner 10, which if not addressed could otherwise result in degradation of the images and/or spectroscopic results obtained during the scanning procedures. For the scanner 10 to operate, however, the shield 6 must still allow communication of data and control signals between the scanner room 1 and the control and equipment rooms 2 and 3, and such communication is generally accomplished through a penetration panel 16.

As shown in FIG. 1A, the penetration panel 16 is typically incorporated into the wall between the scanner and equipment rooms 1 and 3. It features several ports through which the scanner 10 and other devices in the scanner room 1 are connected by cables to the computer console 20 and control subsystems in the control and equipment rooms 2 and 3, respectively. Each port typically includes a filtered BNC connector, which allows the communication of data and/or control signals while still maintaining the barrier to unwanted electromagnetic signals.

Monitoring the vital functions of patients is becoming more common in the MR suite, particularly when the heart or other parts of the cardiovascular system are being scanned. For such cardiac imaging procedures, the patient is often required to remain within the scanner for at least thirty minutes. During this period, it is advisable to monitor the heart, especially if the scanning procedure is being used to diagnose or study cardiac anomalies or whenever the patient is under sedation or anesthesia. Moreover, many cardiovascular imaging protocols involve stimulating the cardiac system, for example, by pharmacological methods, to ascertain how the heart or other parts of the cardiovascular system respond under stress. Again, it is advisable to monitor the heart under these conditions not only to improve the diagnosis but also to improve the safety of the imaging techniques (e.g., to assure that the induced stress to the cardiac system does not approach potentially dangerous levels).

The heart is primary composed of muscle tissue that contracts and relaxes rhythmically to propel blood through the circulatory system of the body. The heartbeat begins with a small nerve bundle located in the upper right-hand corner of the right atrium, an area known as the sinoatrial (SA) node or pacemaker. Cells in the SA node generate electrical impulses at regular intervals of about 60-70 times per minute, though that rate can be increased or decreased by nerves external to the heart that respond to the physiologic demands of the body as well as to other (chemical) stimuli. These impulses travel through and synchronize the rest of the heart, and initiate the depolarization and subsequent repolarization of its muscle, thus causing the heart to contract and relax with a regular, steady beat. This depolarization is distributed from cell to cell, in the form of a wave through the muscle and certain nerve fibers of the heart. Once depolarization is complete, the cardiac cells are able to restore their resting polarity through a process called repolarization. The electrical activity of the heart can be detected through the conductive tissues of the body at the surface by electrodes applied to the skin. A small amount of conductive gel is often applied to the skin, which allows these signals to be more easily transmitted to the electrodes. As shown in FIG. 2, each electrode typically has a metallic detent or connective point to which an electrically conductive leadwire is attached by a corresponding clip. Each leadwire carries a bioelectric signal voltage from its corresponding electrode to an instrument known as an electrocardiograph or to other suitable monitoring equipment. The resulting cardiac signal is derived from the difference in voltages measured as a function of time between two such electrodes. The cardiac signal appears as peaks and valleys in a graphic image known as an electrocardiogram (ECG).

A typical cardiac cycle is illustrated in FIG. 3A. The P segment of the cardiac signal represents the depolarization of the atria. This causes the right atrium to contact and pump venous blood into the right ventricle, and the left atrium to contact and pump oxygenated blood into the left ventricle. The QRS segment denotes the depolarization of cells in the right and left ventricles, causing the pumping of the venous blood to the lungs and the pumping of oxygenated blood to the aorta, respectively. The ST segment of the ECG corresponds to the repolarization or resetting of the electrical system of the heart after the contraction. By observing the form and amplitudes of the segments of the ECG, various cardiac conditions can be diagnosed. The ECG waveform can also be used as a signaling pulse to trigger or synchronize the activity of some other device (e.g., an MR scanner) to the cardiac cycle.

The magnitude and shape of the segments of the ECG signal will vary somewhat depending on the particular lead set used, the exact positions to which the electrodes are attached, and the type or extent of cardiac pathologies. The term “lead” as it pertains to ECG can be used in either the electronic sense (i.e., as a leadwire or cable) or the medical sense (i.e., a spatial position from which the electrical activity of the heart is viewed), which should be clear from the context. For a 3-lead set such as the one shown in FIG. 4, the electrodes would typically be placed on the torso next to the left arm (LA), the right arm (RA), and the left leg (LL) as shown in FIG. 5A, with V_{LA}, V_{RA}, V_{LL} as the potentials measured at those locations. (The potential at the right leg (RL) position {V_{RL}} is not needed for the 3-lead set, but is used as a common reference for other lead set configurations to remove common mode noise.) To obtain ECG data for a 3-lead set, the three leads are defined as:

Lead I=V_{LA}−V_{RA}

Lead II=V_{LL}−V_{RA}

Lead III=V_{LL}−V_{LA}

with each lead representing the difference in potential between two electrodes. Used for standard monitoring of the heart rhythm, the 3-lead set thus requires only three wires to carry the bioelectric signal voltages (one from each of the three electrodes at the RA, LA, and LL positions) to the monitoring equipment. FIG. 3B illustrates the ECG signals obtained from a typical 3-lead set (i.e., one signal from each of Leads I, II and III).

To obtain ECG data for a 6-lead configuration, three augmented leads need to be calculated in addition to Leads I, II, and III. These augmented leads are typically denoted as aVR, aVL, and aVF, and are functions of, and can be derived from, Leads I, II, and III:

aVR=(Lead I+Lead II)/2=(V_{LA}+V_{LL})/2−V_{RA}

aVL=(Lead I−Lead III)/2=V_{LA}−(V_{RA}−V_{LL})/2

aVF=(Lead II+Lead III)/2=V_{LL}−(V_{RA}+V_{LA})/2

No additional electrodes or wire leads are required to obtain the augmented leads because they are functions of the three standard leads (i.e., Leads I, II and III).

In some cases, more information about the electrical activity of the heart is required than even a 6-lead ECG can provide. In these situations, a 12-lead ECG can be used, which requires calculation of six new leads in addition to the augmented leads and the three standard leads. Referred to as precordial leads or chest leads, these six new leads require the use of six additional electrodes positioned at precordial points V₁-V₆ across and along the side of the chest essentially as illustrated in FIG. 5B. Ten electrodes are used overall: the six electrodes positioned at precordial points V₁-V₆ as shown in FIG. 5B and four electrodes at the RA, LA, LL and RL positions as shown in FIG. 5A, with the electrode at the RL position being used as the common reference point. For standard 12-lead ECG recording, at least ten wires must be connected between the patient and the monitoring hardware: three wires to make the connection between the monitor and the electrodes at the RA, LA, and LL positions, six wires to make the connection between the monitor and the electrodes at the precordial points V₁-V₆, and one wire to connect the monitor to the common electrode at the RL position.

The 12-lead configuration measures the electrical activity of the heart not only from multiple perspectives but also with greater detail as to the different phases of the heartbeat. This allows detection of a wider range of cardiac anomalies such as myocardial ischemia (i.e., a decreased flow of blood to the heart that may lead to a heart attack) or myocardial infarction (i.e., death of part of the heart muscle caused by a clot in a coronary artery interrupting the blood supply—a heart attack). Configurations that employ fewer leads, such as the 1-lead or 3-lead arrangements, are generally limited to detecting cardiac arrhythmias (i.e., irregularity in the rhythm or force of the heartbeat).

In an MR suite, ECG monitoring is complicated by the problems associated with the use of electrically conductive wires and electrodes in the electromagnetic environment of the scanner room. According to a well-known phenomenon known as electromagnetic (or Faraday) induction, when a pick-up coil/conductor is exposed to a moving magnetic field, an electrical current is induced in that coil/conductor. In the ECG context, the leadwires, the electrodes, and even the patient's body all tend to act as pick-up coils. The RF pulses and the varying electromagnetic fields generated during an MR scan therefore tend to induce spurious electrical noise in the leadwires. In addition, any movement (e.g., patient breathing) of the leadwires in the varying magnetic fields also tends to induce noise in the wires. Electronic devices commonly found in MR suites, such as fans and lights, may also emit electromagnetic emanations that can induce noise. Furthermore, during an MR procedure, the blood of the patient becomes magnetically polarized due to the magnetic fields and RF pulses. As it flows through the body, the blood generates magnetohydrodynamic potentials that also serve to disrupt and distort the bioelectric signals carried by the electrodes and their associated leadwires. The electrical noise induced in the electrodes and leadwires by all of these sources could ultimately appear as artifacts in the cardiac (ECG) signals displayed by the monitoring equipment.

In addition to the aforementioned noise and motion artifacts, eddy currents can be induced by RF pulses within closed-loop or S-shaped conductors of the type typically found in ECG lead sets. As is well known, eddy currents dissipate their power (voltage times current) as heat within the conductor in which they are induced. In an ECG setting, without the appropriate design safeguards, eddy currents of a magnitude sufficient to cause heating of the leadwires and accompanying electrodes can occur, which can potentially subject the patient to the risk of burn.

Several prior art patents address the problem of noise and motion artifacts in ECG signals. U.S. Pat. No. 5,782,241 to Felblinger et al. discloses a noise reduction technique in which the electrodes and their associated leadwires are somewhat shielded from the electromagnetic environment by being implanted within a shielded housing worn in a belt by the patient. U.S. Pat. Nos. 6,201,981 and 5,511,553 to Yarita and Segalowitz, respectively, propose novel electrode/lead combinations with geometries designed to minimize noise from the electromagnetic fields (by decreasing the effective size of the Faraday pick-up coil). U.S. Pat. No. 6,073,039 to Berson considers solving the noise problem by offering a multi-electrode assembly in which common-mode noise from the electrodes is removed by differential amplification steps after the data acquisition stage. U.S. Pat. No. 5,209,233 to Holland et al. describes the use of passive filtering elements within the electrode/lead set interface to at least partially remove the higher-frequency noise components induced by the scanner from the lower-frequency ECG signal(s).

The problem of eddy current heating is also addressed in the prior art. As alluded to above, eddy-current heating effects arise from the dissipation of power that is derived from the current flow multiplied by the voltage across the leads. Current flow, and hence power (or heat), may be reduced by increasing the resistance of the lead(s) as described in U.S. Pat. No. 5,209,233 to Holland et al., U.S. Pat. No. 4,951,672 to Buchwald et al., U.S. Pat. No. 6,032,063 to Hoar et al., and U.S. Pat. No. 4,280,507 to Rosenberg. The '672 patent, for example, recognizes the need for the leadwires to have a high resistance to reduce the possibility of heating under the electrodes. To that end, it teaches a plurality of resistors in series along the length of the leadwire with the goal of distributing the resistance, and thus avoiding specific hotspots, throughout the length of the leadwire.

The above-cited patents directed to solving the problem of electrode/leadwire heating all embody well-known technological means and are known to be effective in the MR environment. The noise reduction techniques disclosed in the prior art, however, are not as effective or simple to use. In general, the noise induced within an ECG signal by an MR scanner can be an order of magnitude greater than the ECG signal itself, and can exhibit transitions in voltage with very steep rise and fall times. Such steep transitions present high frequency components that are not always readily filterable from the underlying signal by simple hardware components. Because modern MR scanning techniques employ a seemingly ever increasing number of MR (gradient field and RF pulse) sequences, it is not possible to create a single noise reduction system based on simple tuned circuits. A more effective means of general noise reduction would be to acquire a signal derived from the noise signature alone, and then use the information from that noise signature to remove the specific noise component(s) from the corrupted ECG signal. References to the use of noise-specific pick-up coils for use with ECG signal de-noising are found in “Restoration of Electrophysiological Signals Distorted by Inductive Effects of Magnetic Field Gradients During MR Sequences,” by Felblinger et al., MAGNETIC RESONANCE IN MEDICINE 41:715-721 (1999), and “Minimizing Interference from Magnetic Resonance Imagers During Electrocardiography,” by Laudon et al., IEEE TRANSACTIONS ON BIOMEDICAL ENGINEERING,” 45:160-164 (FEB. 1998). These two references, as with the patents cited above, are incorporated herein by reference.

Due to the rapid developments in functional and interventional MRI, there is an increasing need to obtain noise-free electrophysiological signals of all types (e.g., electro-oculograms [EOGs], electroencephalograms [EEGs], and electromyograms [EMGs]) not just ECG signals. These signals are also greatly perturbed by MR sequences, particularly the faster ones, which make such corporeal signals very difficult to interpret. For patient monitoring during interventional MRI or even for examinations of premature babies or anesthetized patients, a noise-free ECG is also needed to complement other physiologic measurements such as blood pressure, respiration rate, pulse oximetry, and temperature. Due to the shape of its QRS segment, an artifact-free ECG signal is better defined than the signal from a pulse oximeter and therefore is preferred for triggering and patient monitoring. A reliable ECG is thus particularly important given its routine use as the starter or trigger of MR sequences.

The need for uncorrupted corporeal signals has thus spurred efforts to develop more effective techniques of preventing, removing or otherwise overcoming noise and motion artifacts that harsh electromagnetic environments, such as MR suites, typically superimpose on such signals. It would therefore be quite advantageous to develop such noise-reduction technique(s) and incorporate them, along with heat-reduction techniques such as those noted above, into lead set and related technology. For example, no known ECG or other type of lead set assembly to date incorporates heat-reduction techniques with an embedded noise-sensing loop in the same device.

### SUMMARY OF THE INVENTION

Several objectives and advantages of the invention are attained by the various embodiments and related aspects of the invention summarized below.

In a presently preferred embodiment, the invention provides a lead set assembly for interconnecting a plurality of electrodes and a separate device. The electrodes are of the type that attach to a subject for receiving bioelectric signals therefrom. The lead set assembly comprises a condition sense lead and a noise pickup lead. The condition sense lead includes a pair of conductive paths, each of which being connectible to one of the electrodes for conveying a bioelectric signal therefrom and capable of having a first noise signal induced therein by electromagnetic emanations. The noise pickup lead also includes a pair of conductive paths, each of which being effectively topographically aligned with a corresponding one of the conductive paths of the condition sense lead to form a noise-matched pair therewith and capable of having a second noise signal induced therein by the electromagnetic emanations. In each of the noise-matched pairs, this enables the second noise signal to be substantially approximate to the first noise signal. The lead set assembly can thus be used to convey from the electrodes to the separate device (I) the bioelectric and first noise signals using the conductive paths of the condition sense lead and (II) the second noise signals using the conductive paths of the noise pickup lead.

In a related embodiment, the invention provides a lead set assembly whose condition sense and noise pickup leads may each be configured with two or more pairs of conductive paths.

The invention also provides a method of ascertaining the condition of a patient. The method includes the step of removably associating a plurality of electrodes with the patient, with each of the electrodes being used to pickup a bioelectric signal indicative of the patient's condition. The method also involves connecting a lead set assembly between the electrodes and a separate device. The lead set assembly includes a condition sense lead and a noise pickup lead. The condition sense lead features a pair of conductive paths, each of which being connectible to one of the electrodes for conveying a bioelectric signal therefrom and capable of having a first noise signal induced therein by electromagnetic emanations. The noise pickup lead also features a pair of conductive paths, each of which being effectively topographically aligned with one of the conductive paths of the condition sense lead to form a noise-matched pair therewith and capable of having a second noise signal induced therein by the electromagnetic emanations. In each noise-matched pair, this enables the second noise signal to be substantially approximate to the first noise signal. The lead set assembly can thus be used to convey to the separate device (I) the bioelectric and first noise signals using the conductive paths of the condition sense lead and (II) the second noise signals using the conductive paths of the noise pickup lead. The method also includes the step of monitoring the condition of the patient through use of the bioelectric signals received by the separate device.

The invention further provides a magnetic resonance (MR) system. The MR system includes a means for controlling gradient and radio frequency (RF) coils to apply changing magnetic fields and RF signals to a patient to induce and encode magnetic resonance within selected nuclei therein. It also features a means for receiving magnetic resonance signals from the selected nuclei in response thereto and processing the magnetic resonance signals into diagnostic information. The MR system further includes a plurality of electrodes and a lead set assembly. The electrodes are removably attachable to the patient, with each being used to pickup a bioelectric signal indicative of the patient's condition. The lead set assembly comprises a condition sense lead and a noise pickup lead. The condition sense lead includes a pair of conductive paths, each of which being connectible to one of the electrodes for conveying a bioelectric signal therefrom and capable of having a first noise signal induced therein by electromagnetic emanations. The noise pickup lead also includes a pair of conductive paths, each of which being effectively topographically aligned with a corresponding one of the conductive paths of the condition sense lead to form a noise-matched pair therewith and capable of having a second noise signal induced therein by the electromagnetic emanations. In each of the noise-matched pairs, this enables the second noise signal to be substantially approximate to the first noise signal. The lead set assembly can thus be used to convey from the electrodes to the separate device (I) the bioelectric and first noise signals using the conductive paths of the condition sense lead and (II) the second noise signals using the conductive paths of the noise pickup lead.

### BRIEF DESCRIPTION OF THE DRAWINGS

The invention, and particularly its presently preferred and alternative embodiments and related aspects, will be better understood by reference to the detailed disclosure below and to the accompanying drawings, in which:

FIG. 1A illustrates the layout of an MR suite inclusive of the scanner room in which the scanner and patient table are located, the control room in which the computer console for controlling the scanner is situated, and the equipment room in which various control subsystems for the scanner are sited.

FIG. 1B shows a scanner and table of the type shown schematically in FIG. 1A.

FIG. 1C is a more detailed view of the MR system shown in FIGS. 1A and 1B showing the computer console and the various subsystems in the control and equipment rooms and a cross-section of the scanner and table situated in the scanner room.

FIG. 2 illustrates a prior art leadwire of the type typically used to convey bioelectric signals from an electrode attached to the skin of a patient to suitable monitoring equipment such as an ECG monitor.

FIG. 3A illustrates an example of a typical cardiac cycle or ECG signal on which the specific segments of the ECG waveform are designated.

FIG. 3B illustrates an example of the ECG signals obtainable from a lead set assembly having at least 3 leadwires (i.e., one signal from each of Leads I, II and III).

FIG. 4 illustrates a 3-lead lead set assembly of the type capable of carrying the bioelectric signals used to produce the ECG signals shown in FIG. 3B.

FIG. 5A illustrates a torso of a human body and the various positions to which electrodes can be attached to obtain the ECG signals shown in FIG. 3B using, for example, the 3-lead lead set assembly shown in FIG. 4.

FIG. 5B illustrates a torso of a human body and the six precordial points V₁-V₆ thereon to which six additional electrodes can be attached to obtain a 12-lead ECG recording.

FIG. 6 is a top view of a lead set assembly according to a presently preferred embodiment of the invention, showing the conductive paths of both the condition sense lead and the noise pickup lead.

FIGS. 7A and 7B illustrate the lead set assembly of FIG. 6, showing its condition sense lead and its noise pickup lead separated for purposes of illustration.

FIG. 8 is a partial, exploded cross-sectional view of the lead set assembly shown in FIG. 6.

FIG. 9 shows the lead set assembly of FIG. 6 in which the conductive paths of the condition sense have their impedances determined by the geometry, topology, and/or layout of those paths, their composition, and/or the composition of adjacent layers.

FIG. 10 shows another variation of the lead set assembly shown in FIG. 6 in which the conductive paths of the condition sense lead have their impedances determined by inline impedance controlling elements incorporated into the conductive paths.

FIGS. 11A and 11B illustrate one way of incorporating inline impedance controlling elements within the conductive paths of the condition sense lead and noise pickup lead, respectively, of the lead set assembly of FIG. 10.

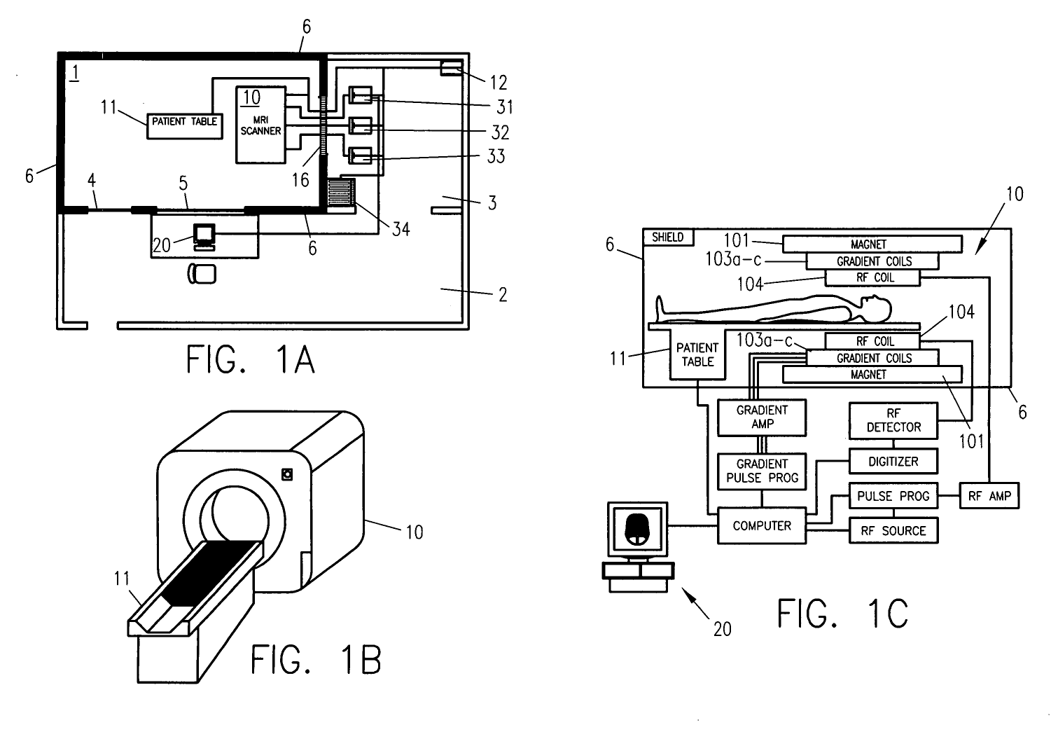

### DETAILED DESCRIPTION OF THE INVENTION AND ITS PREFERRED EMBODIMENTS AND RELATED ASPECTS

Although the invention herein described and illustrated is presented primarily in the context of ECG lead set assemblies designed for use in the MR environment, the reader will understand that the invention can be applied or adapted not only to a wide variety of other lead set applications (e.g., EEGs and EMGs) but also to various other types of noisy environments. The presently preferred embodiment and related aspects of the invention will now be described with reference to the accompanying drawings, in which like elements have been designated where possible by the same reference numerals.

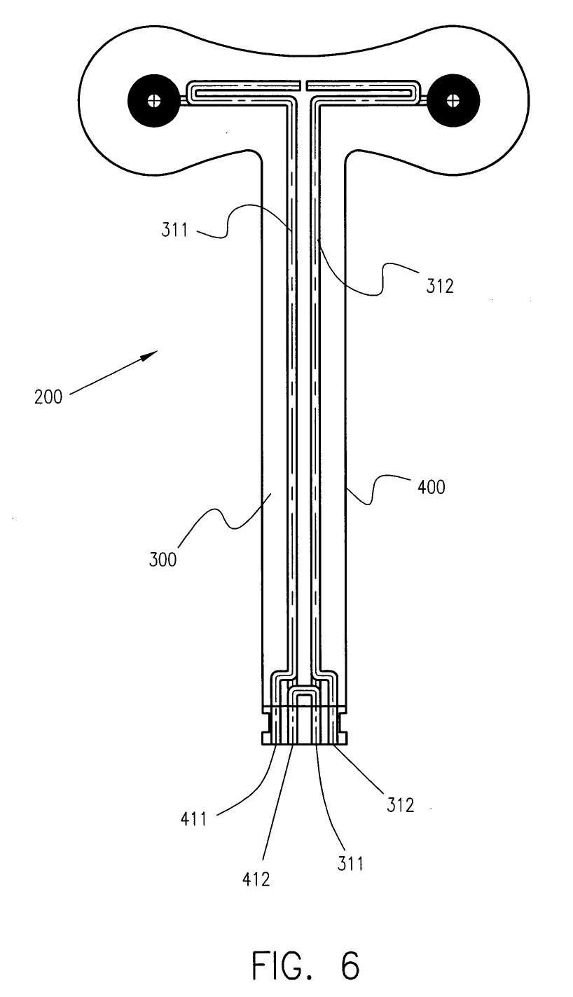

FIGS. 6-8 illustrate one presently preferred embodiment of the invention, namely, a lead set assembly, generally designated 200, for use in conveying bioelectric signals of the type used in forming cardiac (ECG) signals. Like all of the other embodiments disclosed herein, lead set assembly 200 is particularly well adapted for use within noisy environments. In an MR environment, for example, lead set assembly 200 and the patient to whom it is connected will be subject to electromagnetic emanations primarily composed of static magnetic fields, varying magnetic fields and radio frequency (RF) signals generated during the course of an MR procedure. Such electromagnetic emanations can also include the electrical noise generated by the various electrical and electronic devices typically found in MR suites, such as injectors, pumps, patient monitors, fans, music systems, and various types of communications equipment.

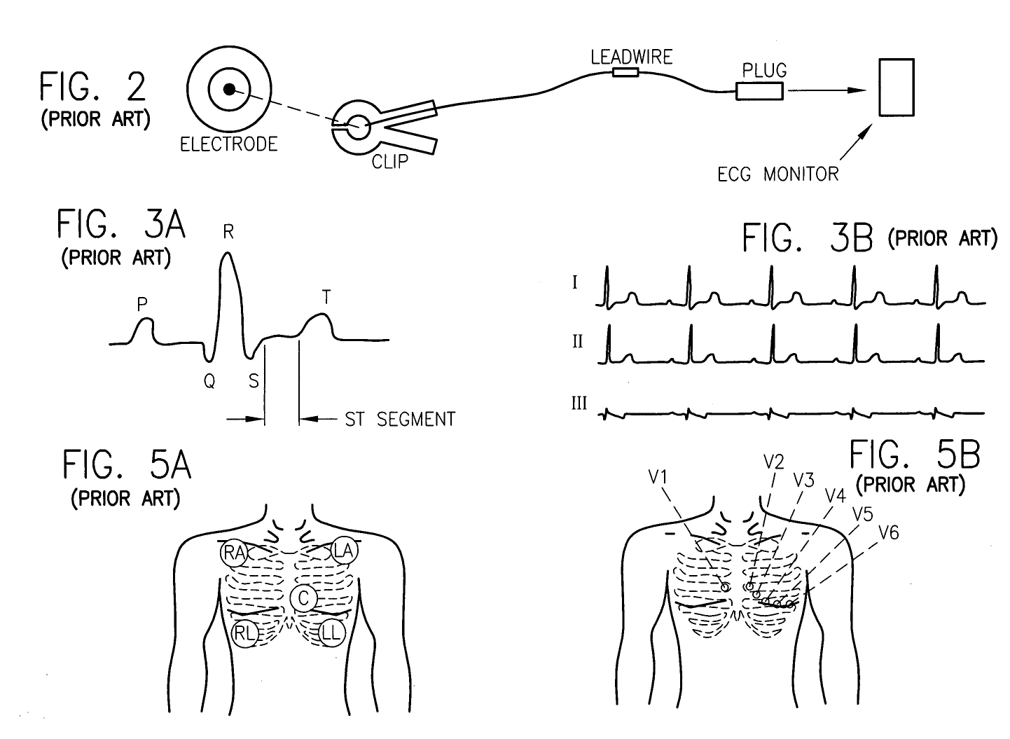

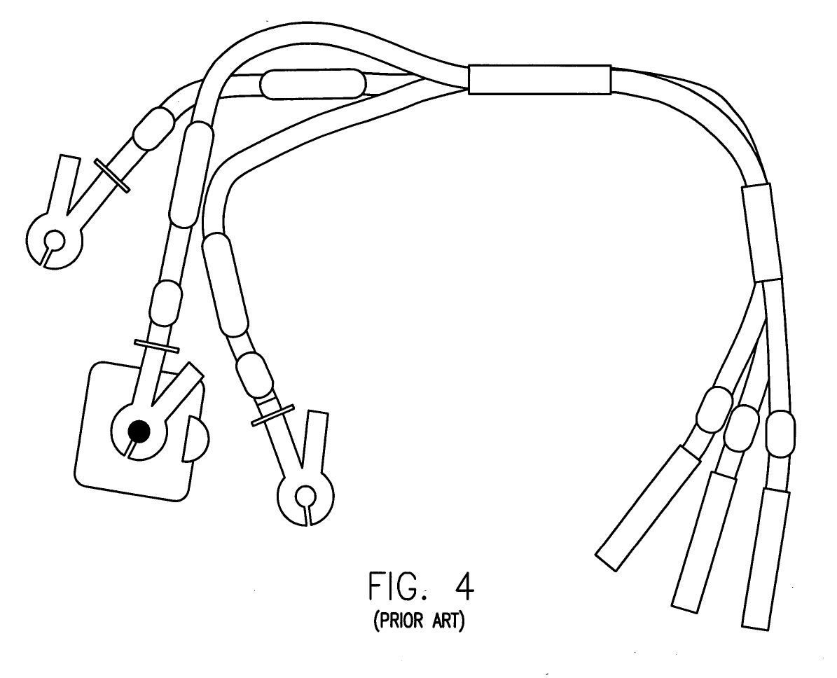

As shown in FIG. 6, lead set assembly 200 can be used to interconnect two electrodes and a separate device. Alternatively, instead of using electrodes provided separately, the two electrodes may be provided as part of the lead set assembly 200 itself. The electrodes capable of being used with this invention are of the type that attach or are otherwise associable with the patient for receiving bioelectric signals therefrom. FIG. 2 shows an example of the type of electrode suitable for use with the present invention.

The lead set assembly 200 includes a condition sense lead 300 and a noise pickup led 400. The condition sense lead 300 includes a pair of conductive paths 311 and 312, each of which connectible to one electrode for conveying a bioelectric signal therefrom. In addition, each conductive path is also susceptible to having a first noise signal induced therein by whatever electromagnetic emanations to which it may be exposed. The noise pickup lead 400 also includes a pair of conductive paths 411 and 412, each of which being effectively topographically aligned with one of the conductive paths of the condition sense lead 300 so as to form a noise-matched pair or set therewith. These conductive paths 411 and 412 are also each capable of having noise induced therein, referred to herein as second noise signals. Whether or not such noise signals are actually induced within the conductive paths 311, 312, 411 and 412 will depend, of course, on several factors including the strength of the electromagnetic emanations and the related issue of the proximity of the lead set assembly 200 to source(s) of noise.

The two conductive paths 311 and 312 of condition sense lead 300 are preferably matched as closely as possible. (The same thing applies to the conductive paths of noise pickup lead 400.) Although intended to convey the electrical potentials detected by the two electrodes, the conductive paths also tend to behave like receiving antennas. Each conductive path will thus pickup some noise due to the RF pulses, magnetic fields and/or other electromagnetic emanations encountered within the MR suite. How well or poorly the conductive paths operate as antennas can be affected, however, by their impedance.

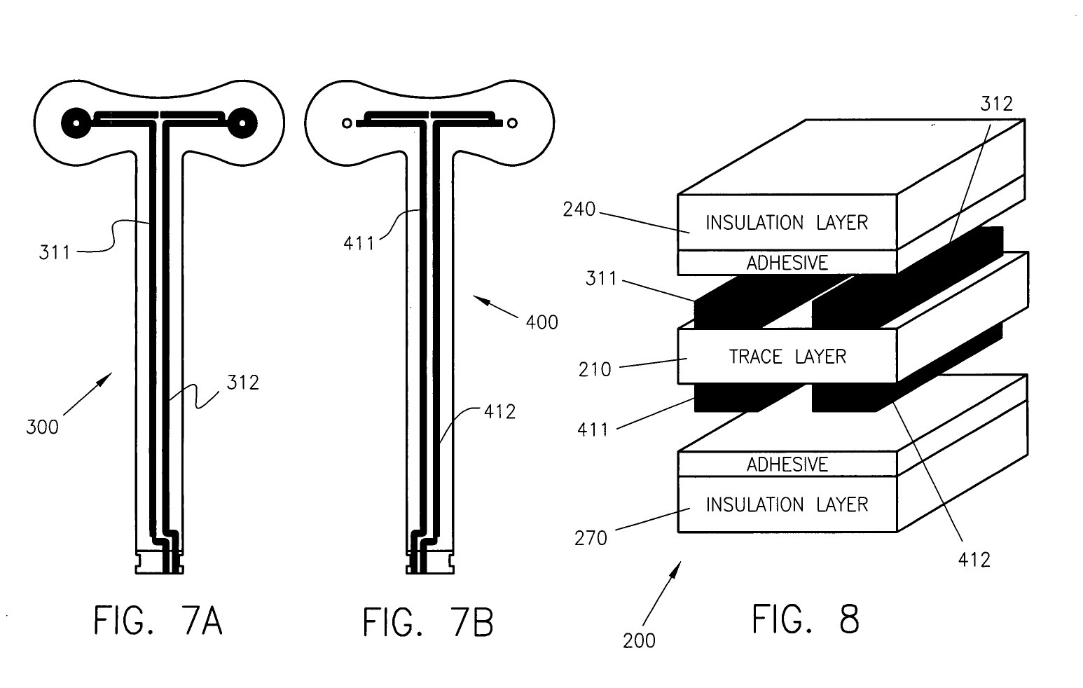

Referring still to the preferred embodiment shown in FIG. 6, the condition sense lead 300 will be used to convey two electrical (voltage) signals to the separate device, possibly through intermediate signal conditioning circuitry (e.g., an op-amp, etc.). In the case of an ECG lead set assembly, for example, the condition sense lead 300 will be used to convey two bioelectric signals indicative of cardiac activity to, ultimately, an ECG monitor such as shown in FIG. 2 or other monitoring, diagnostic or control equipment. As best shown in FIG. 7A, one electrical signal p_{C1} will be the output from the first conductive path 311, and the other electrical signal p_{C2} will be the output of the second conductive path 312.

Consider p_{C1}. It can be represented by p_{C1}=s₁+n₁, where si is the electric potential (bioelectric signal) picked up by the first electrode and n_{C1} is the first noise signal induced within its corresponding conductive path 311. Similarly, the voltage signal p_{C2} can be represented by p_{C2}=s₂+n_{C2}, where s₂ is the electric potential (bioelectric signal) from the second electrode and n_{C2} is the first noise signal induced within its corresponding conductive path 312. The bioelectric signals s₁ and s₂ will, of course, be quite low in magnitude because the electric potentials to be picked up by the electrodes are well known to be quite weak. Absent noise pickup lead 400 discussed below, the noise signals induced within the conductive paths 311 and 312 of condition sense lead 300 would otherwise make detection of the bioelectric signals s₁ and s₂ more difficult.

As noted in background, it is well known that the cardiac cycle or signal of a patient can be obtained from the difference in potentials represented by the two bioelectric signals s₁ and s₂. Ideally, this can be represented by the equation:

v=s₁−s₂ (1)

In practice, however, superimposed on the bioelectric signal s₁ picked up by the first electrode will be the first noise signal e_{C1} induced within conductive path 311. Similarly, the first noise signal n_{C2} induced within the other conductive path 312 will be superimposed on the bioelectric signal s₂ detected by the second electrode. The actual outputs provided by the conductive paths 311 and 312 of condition sense lead 300 are thus represented, respectively, by:

p_{C1}=s₁+n_{C1} (1)

p_{C2}=s₂+n_{C2.} (3)

Therefore, the cardiac signal must be ascertained from:

$$( 4 ) \begin{matrix}\overline{v} = {p}_{C1} - {p}_{C2} \\ = ( {s}_{1} + {n}_{C1} ) - ( {s}_{2} + {n}_{C2} ) \\ = ( {s}_{1} - {s}_{2} ) + ( {n}_{C1} - {n}_{C2} ) .\end{matrix}$$

Because the bioelectric signals s₁ and s₂ can be essentially masked or overwhelmed by the noise signals n_{C1} and n_{C2}, provision must be made to render the bioelectric signals detectable and usable by whatever device to which lead set assembly 200 is connected. In the present invention, this is accomplished at least in part by matching as closely as possible the impedance of the conductive path 311 for the first electrode with that of the conductive path 312 for the second electrode. Doing so will make the first noise signal n_{C1} induced within the conductive path 311 for the first electrode as similar as possible to the first noise signal n_{C2} induced within the conductive path 312 for the second electrode.

Absent differential noise pickup at the electrodes themselves, if the conductive paths 311 and 312 are produced according to suitably strict manufacturing tolerances, the condition sense lead 300 will be able to provide noise signals that are essentially equal. With n_{C1}=n_{C2}, equation (4) then becomes:

\overline{v}≅s₁−s₂, (5)

which in view of equation (1) yields:

\overline{v}≅v=s₁−s₂. (6)

Neglecting noise sources at the patient/electrode interface, equations (5) and (6) demonstrate the importance of matching the impedance of the two conductive paths 311 and 312 for the condition sense lead 300. The cardiac signal of a patient can thus be more readily obtained from the condition sense lead as long as its first and second conductive paths 311 and 312 are appropriately impedance matched.

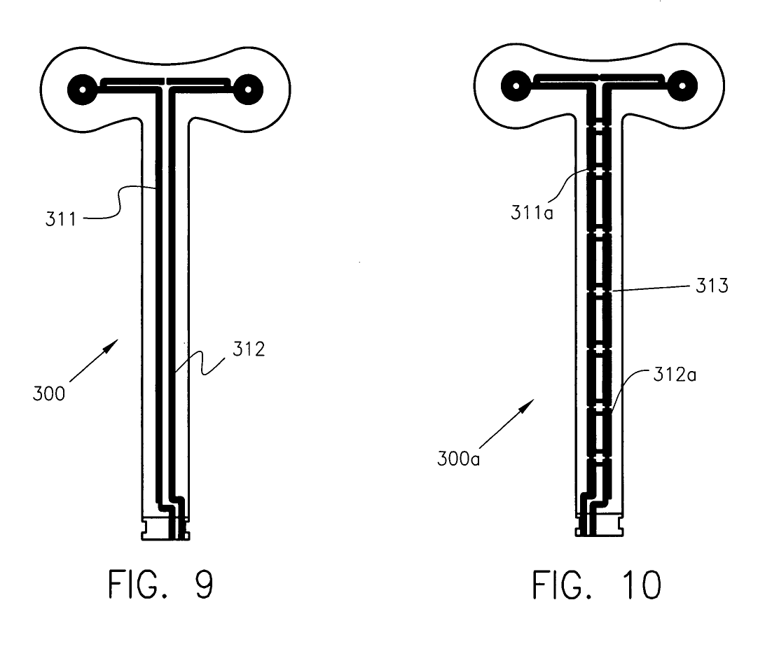

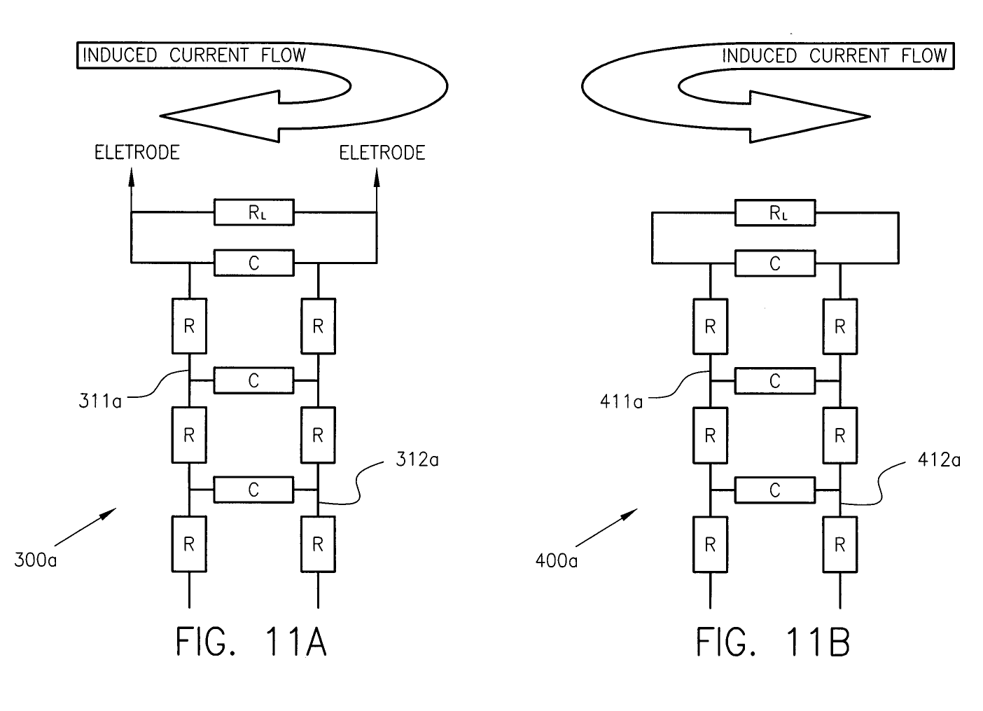

The impedance of condition sense lead 300, and more specifically of its conductive paths, can be implemented at least partially through the geometry of the conductive paths, the topography of the conductive paths, the composition of the conductive paths, and/or the composition of the adjacent layers. This is best illustrated in FIG. 9. Alternatively, the impedance of the condition sense lead could be implemented at least partially through inline impedance controlling elements incorporated into gap(s) 313 in the conductive paths as shown in FIG. 10. This is best shown in FIG. 11A in which, in this variation of lead set assembly 200, the condition sense lead is designated as 300a and its first and second conductive paths are designated as 311a and 312b, respectively. The inline impedance controlling elements can take the form of resistors, capacitors and inductors or any combination or number thereof.

One constraint on the impedance of the condition sense lead 300/300a, however, is that it must be greater than the impedance of the electrode/patient interface to avoid radio-frequency induced eddy-current heating therein that could otherwise pose a risk of burn to the patients. This can be accomplished at least in part by use of a first impedance load connected between the conductive paths 311/311a and 312/312a, as is best shown in FIG. 11A. Although this load is shown as a resistor R_{L} and a capacitor C, it could also be implemented with an inductor or any combination of those three circuit elements.

In actual practice, the noise signals n_{C1} and n_{C2} induced within the conductive paths of the condition sense lead will likely not be precisely equal due to mismatched impedance of the conductive paths plus the additional noise sources. Consequently, instead of the result suggested by equations (5) and (6), equation (4) will more likely yield the following relationship from which the cardiac signal will have to be ascertained:

\overline{v}=(s₁−s₂)+(n_{C1}−n_{C2}) (7)

where, as noted above, n_{C1}≠n_{C2}.

The lead set assembly 200 therefore includes noise pickup lead 400, which is preferably disposed either beneath or atop the condition sense lead. As discussed in connection with the condition sense lead, the impedance of the two conductive paths 411 and 412 of noise pickup lead 400 should also be matched as closely as possible. The impedance of noise pickup lead 400 can therefore be implemented at least partially through the geometry, topography and composition of the conductive paths, and/or the composition of the adjacent layers. The impedance of the noise pickup lead could also be implemented at least partially through inline impedance controlling elements as noted above with respect to the variation of condition sense lead 300 (i.e., 300a). This particular variation is best shown in FIG. 11B as noise pickup lead 400a.

Unlike those in the condition sense lead, however, these two conductive paths 411 and 412 are designed to act only as receiving antennas, as they are intended to pickup noise only and not carry bioelectric signals. The output from the first conductive path 411 can thus be represented by p_{N1}=n_{N1}, where n_{N1} is the noise induced within that path by the electromagnetic emanations. Similarly, the output of the second conductive path 412 can be represented by p_{N2}=n_{N2}, where n_{N2} represents the noise induced. For purposes that will soon become apparent, n_{N1} and n_{N2} are also referred to herein as second noise signals. As best shown in FIG. 7B, the outputs provided by the first and second conductive paths 411 and 412 of the noise pickup lead are thus represented, respectively, by:

p_{N1}=n_{N1} (8)

p_{N2}=n_{N2.} (9)

As noted above, each conductive path of noise pickup lead 400 is preferably topographically aligned with one of conductive paths of condition sense lead 300 to form a noise-matched pair or set therewith. More specifically, the first conductive path 411 of the noise pickup lead and the first conductive path 311 of the condition sense lead form one noise-matched set, with the second conductive paths 411 and 311 of the noise pickup and condition sense leads, respectively, forming the other noise-matched set. The impedances of conductive paths 311 and 411 should therefore be matched as closely as possible, as should the impedances of conductive paths 312 and 412. A constraint on the impedance of the noise pickup lead 400, however, is that it should be substantially equal to the impedance of the electrode/patient interface. This can be accomplished at least in part by use of another impedance load connected between the conductive paths of the noise pickup lead. This is best shown in FIG. 11B. Although this load is shown as a resistor R_{L} and a capacitor C, it could also be implemented with an inductor or any combination of those three circuit elements.

Given the identical or at least substantially similar topologies of the conductive paths of each noise-matched pair/set, the second noise signal induced within each conductive path of the noise pickup lead should be substantially approximate to the first noise signal induced within the corresponding conductive path of the condition sense lead. More specifically, the second noise signal n_{N1} induced within the first conductive path 411/411a of the noise pickup lead should be substantially approximate to the first noise signal n_{C1} induced within the first conductive path 311/311a of the condition sense lead. Similarly, the second and first noise signals n_{N2} and n_{C2} induced within the second conductive paths 412/412a and 312/312a of the noise pickup and condition sense leads 400/400a and 300/300a, respectively, should also be substantially approximate.

The outputs of lead set assembly 200 can be made available to whatever device to which the lead set assembly is connected. As alluded to above, this could be a signal conditioning circuit, signal processing circuit, an ECG monitor, or other diagnostic or control equipment. Regardless of which separate device to which the lead set assembly will be connected, that separate device shall necessarily include some means for readily accepting the outputs of the lead set assembly. It shall also embody some method of conditioning, processing or otherwise using those outputs in such a way to remove, cancel or otherwise eliminate the noise signals from the underlying bioelectric signals. Such a method would provide substantially noise-free bioelectric signals which could then used in any number of ways either unknown or known in the relevant arts.

As shown in FIG. 8, the conductive paths of the condition sense and noise pickup leads are preferably attached to opposite sides, respectively, of an inner insulating layer 210 and sandwiched between outer insulating layers 240 and 270. These layers are preferably flexible and transparent to and non-interfering with respect to the functions of the MR scanner. Whatever material(s) out of which the insulating layers are made, it/they should be chosen specifically for its/their dielectric properties and perhaps as well as for controlling the impedance of the conductive paths. Various nonconductive materials such as polyester, Kapton, and/or Teflon are suitable for this purpose.

The conductive paths should also be flexible and transparent to and non-interfering with the functions of the MR scanner, and their composition may be chosen so that it is part of the overall design for control of the impedance of the conductive paths themselves. They are ideally made of a carbon based material, preferably conductive graphite. They can be attached to the inner insulating layer 210 by any means appropriate to the materials, including but not limited to drawing, inscribing, imbedding, and/or other lithographic-type process. The conductive paths may be attached to the outer insulating layers 240 and 270 by an adhesive, as is also shown in FIG. 8.

As best shown in FIGS. 6, 7A and 8, the conductive paths of condition sense lead 300 each follow a looping path on the top side of insulating layer 210 that extends from its point of attachment for its corresponding electrode to a location to which a connector may be attached. FIGS. 6, 7B and 8 together show that the conductive paths 411 and 412 of noise pickup lead 400 follow the same path as conductive paths 311 and 312 but on the underside of inner insulating layer 210, without connecting to the electrodes and in no place making electrical contact with the conductive paths of the condition sense lead.

For the condition sense and noise pickup leads 300a and 400a whose impedances are affected using the inline impedance controlling elements, the outer insulating layers 240 and 270 protect their conductive paths from exterior contact except at specific locations. Such locations include the gaps 313, as shown in FIG. 10, where the inline components would be attached to the conductive paths and the spaces across which the conductive paths are spanned by the two impedance loads as shown in FIGS. 11A and 11B. The area near the connector with which the lead set assembly would be coupled to the separate device(s) noted above should also appropriately protected.

In a related embodiment, the present invention provides a lead set assembly whose condition sense and noise pickup leads may each be configured with two or more pairs of conductive paths. It should be apparent from the foregoing that each lead should preferably have an impedance load connected across each of its pairs of conductive paths, consistent with the impedance loads discussed above in connection with FIGS. 11A and 11B. Furthermore, the condition sense and noise pickup leads in this embodiment can also have their impedances controlled in the same way as that suggested by FIGS. 9 and 10 and the text above corresponding thereto. Lastly, the additional outputs of this lead set assembly would also have to be accommodated by whatever device to which the lead set assembly is connected. The separate device not only shall readily accept the additional outputs but also shall embody some method of conditioning, processing or otherwise using those additional outputs in such a way to remove, cancel or otherwise eliminate the noise signals from the underlying bioelectric signals.

The presently preferred and alternative embodiments for carrying out the invention have been set forth in detail according to the Patent Act. Persons of ordinary skill in the art to which this invention pertains may nevertheless recognize alternative ways of practicing the invention without departing from the spirit of the following claims. Consequently, all changes and variations that fall within the literal meaning, and range of equivalency, of the claims are to be embraced within their scope. Persons of such skill will also recognize that the scope of the invention is indicated by the claims below rather than by any particular example or embodiment discussed or shown in the foregoing description.

Accordingly, to promote the progress of science and useful arts, we secure for ourselves by Letters Patent exclusive rights to all subject matter embraced by the following claims for the time prescribed by the Patent Act.

What is claimed is:

1. A lead set assembly for interconnecting a plurality of electrodes and a separate device, said plurality of electrodes of a type for attaching to a subject for receiving bioelectric signals therefrom, the lead set assembly comprising:
(a) a condition sense lead having a pair of conductive paths, each of said conductive paths being connectible to one of said electrodes for conveying a bioelectric signal therefrom and capable of having a first noise signal induced therein by electromagnetic emanations; and
(b) a noise pickup lead having a pair of conductive paths each of which being effectively topographically aligned with a corresponding one of said conductive paths of said condition sense lead to form a noise-matched pair therewith and capable of having a second noise signal induced therein by the electromagnetic emanations, thereby enabling said second noise signal to be substantially approximate to said first noise signal in each of said noise-matched pairs, said lead set assembly thus for conveying from said electrodes to said separate device (I) said bioelectric signals and said first noise signals using said conductive paths of said condition sense lead and (II) said second noise signals using said conductive paths of said noise pickup lead.

2. The lead set assembly of claim 1 wherein the difference in voltages of said bioelectric signals conveyed from said electrodes by said conductive paths of said condition sense lead yields a corporeal signal.

3. The lead set assembly of claim 2 wherein said corporeal signal is a cardiac signal.

4. The lead set assembly of claim 1 wherein said condition sense lead is disposed one of (i) atop said noise pickup lead and (ii) beneath said noise pickup lead.

5. The lead set assembly of claim 1 wherein said condition sense lead and said noise pickup lead are insulated from each other with nonconductive film.

6. The lead set assembly of claim 1 wherein said conductive paths of said condition sense lead and said noise pickup lead are composed of a carbon based material.

7. The lead set assembly of claim 6 wherein said carbon based material includes conductive graphite.

8. The lead set assembly of claim 1 wherein said conductive paths of said condition sense lead and said noise pickup lead are attached to opposite sides, respectively, of an inner insulating layer and sandwiched between outer insulating layers.

9. The lead set assembly of claim 8 wherein said conductive paths of said condition sense lead and said noise pickup lead are attached to said inner insulating layer by at least one of drawing, inscribing, imbedding and lithography.

10. The lead set assembly of claim 8 wherein said conductive paths of said condition sense lead and said noise pickup lead are attached to said outer insulating layers by an adhesive.

11. The lead set assembly of claim 8 wherein said inner and said outer insulating layers are each made of nonconductive film.

12. The lead set assembly of claim 1 wherein said conductive paths of said condition sense lead have impedances that are substantially identical.

13. The lead set assembly of claim 1 wherein said conductive paths of said noise pickup lead have impedances that are substantially identical.

14. The lead set assembly of claim 1 wherein said conductive paths of each of said noise-matched pairs have impedances that are substantially identical so that said first and said second noise signals induced therein are substantially approximate.

15. The lead set assembly of claim 1 wherein said condition sense lead and said noise pickup lead have impedances that are substantially identical.

16. The lead set assembly of claim 1 wherein an impedance of at least one of said condition sense and said noise pickup leads is implemented at least partially through at least one of (i) a geometry of said conductive paths, (ii) a topography of said conductive paths, (iii) a composition of said conductive paths, and (iv) a composition of adjacent layers,

17. The lead set assembly of claim 1 wherein an impedance of at least one of said condition sense lead and said noise pickup lead is implemented at least partially through inline impedance controlling elements incorporated into at least one of said conductive paths thereof.

18. The lead set assembly of claim 17 wherein said inline impedance controlling elements include at least one of a resistor, a capacitor and an inductor.

19. The lead set assembly of claim 1 wherein at least one of said conductive paths defines at least one gap therein across each of which an impedance controlling element is attached for enabling at least one of said condition sense lead and said noise pickup lead to be tuned to a desired impedance.

20. The lead set assembly of claim 1 further including a first impedance load interconnecting said conductive paths of said condition sense lead, said first impedance load enabling said condition sense lead to have an impedance greater than that existing at an interface between said electrodes and said subject.

21. The lead set assembly of claim 20 wherein said first impedance load includes at least one of a resistor, a capacitor and an inductor.

22. The lead set assembly of claim 1 further including a second impedance load interconnecting said conductive paths of said noise pickup lead, said second impedance load enabling said noise pickup lead to have an impedance substantially equal to that existing at an interface between said electrodes and said subject.

23. The lead set assembly of claim 22 wherein said second impedance load includes at least one of a resistor, a capacitor and an inductor.

24. The lead set assembly of claim 1 further including a connector by which the lead set assembly, and said conductive paths thereof, can be coupled to said separate device.

25. A lead set assembly for coupling a plurality of electrodes to a separate device, said plurality of electrodes of a type for attaching to a subject for receiving bioelectric signals therefrom, the lead set assembly comprising:
(a) a condition sense lead having first and second conductive paths, said first and said second conductive paths each being connectible to one of said electrodes for conveying a bioelectric signal therefrom and capable of having a first noise signal induced therein by electromagnetic emanations; and
(b) a noise pickup lead having (I) a first conductive path being effectively topographically aligned with said first conductive path of said condition sense lead to form a first noise-matched pair therewith and capable of having a second noise signal induced therein by the electromagnetic emanations and (II) a second conductive path being effectively topographically aligned with said second conductive path of said condition sense lead to form a second noise-matched pair therewith and capable of having a second noise signal induced therein by the electromagnetic emanations, thereby enabling said second noise signal to be substantially approximate to said first noise signal in each of said noise-matched pairs, said lead set assembly thus for conveying from said electrodes to said separate device (I) said bioelectric signals and said first noise signals using said conductive paths of said condition sense lead and (II) said second noise signals using said conductive paths of said noise pickup lead.

26. The lead set assembly of claim 25 wherein:
(a) said condition sense lead also has third and fourth conductive paths, said third and said fourth conductive paths each being connectible to an other one of said electrodes for conveying a bioelectric signal therefrom and capable of having a first noise signal induced therein by the electromagnetic emanations; and
(b) said noise pickup lead also has (I) a third conductive path being effectively topographically aligned with said third conductive path of said condition sense lead to form a third noise-matched pair therewith and capable of having a second noise signal induced therein by the electromagnetic emanations and (II) a fourth conductive path being effectively topographically aligned with said fourth conductive path of said condition sense lead to form a fourth noise-matched pair therewith and capable of having a second noise signal induced therein by the electromagnetic emanations, thereby enabling said second noise signal to be substantially approximate to said first noise signal in each of said third and said fourth noise-matched pairs, said lead set assembly thus also for conveying from said other electrodes to said separate device (I) said bioelectric signals and said first noise signals using said third and said fourth conductive paths of said condition sense lead and (II) said second noise signals using said third and said fourth conductive paths of said noise pickup lead.

27. The lead set assembly of claim 26 wherein the difference in voltages of:
(a) said bioelectric signals conveyed from said electrodes by said first and said second conductive paths of said condition sense lead yields a first corporeal signal; and
(a) said bioelectric signals conveyed from said electrodes by said third and said fourth conductive paths of said condition sense lead yields a second corporeal signal.

28. The lead set assembly of claim 27 wherein said first and said second corporeal signals are cardiac signals.

29. The lead set assembly of claim 26 wherein said condition sense lead is disposed one of (i) atop said noise pickup lead and (ii) beneath said noise pickup lead.

30. The lead set assembly of claim 26 wherein said condition sense lead and said noise pickup lead are insulated from each other with nonconductive film.

31. The lead set assembly of claim 26 wherein said conductive paths of said condition sense lead and said noise pickup lead are composed of a carbon based material.

32. The lead set assembly of claim 31 wherein said carbon based material includes conductive graphite.

33. The lead set assembly of claim 26 wherein said conductive paths of said condition sense lead and said noise pickup lead are attached to opposite sides, respectively, of an inner insulating layer and sandwiched between outer insulating layers.

34. The lead set assembly of claim 33 wherein said conductive paths of said condition sense lead and said noise pickup lead are attached to said inner insulating layer by at least one of drawing, inscribing, imbedding and lithography.

35. The lead set assembly of claim 33 wherein said conductive paths of said condition sense lead and said noise pickup lead are attached to said outer insulating layers by an adhesive.

36. The lead set assembly of claim 33 wherein said inner and said outer insulating layers are each made of nonconductive film.

37. The lead set assembly of claim 26 wherein said conductive paths of said condition sense lead have impedances that are substantially identical.

38. The lead set assembly of claim 26 wherein said conductive paths of said noise pickup lead have impedances that are substantially identical.

39. The lead set assembly of claim 26 wherein said conductive paths of each of said noise-matched pairs have impedances that are substantially identical so that said first and said second noise signals induced therein are substantially approximate.

40. The lead set assembly of claim 26 wherein said condition sense lead and said noise pickup lead have impedances that are substantially identical.

41. The lead set assembly of claim 26 wherein an impedance of at least one of said condition sense and said noise pickup leads is implemented at least partially through at least one of (i) a geometry of said conductive paths, (ii) a topography of said conductive paths, (iii) a composition of said conductive paths, and (iv) a composition of adjacent layers.

42. The lead set assembly of claim 26 wherein an impedance of at least one of said condition sense lead and said noise pickup lead is implemented at least partially through inline impedance controlling elements incorporated into at least one of said conductive paths thereof.

43. The lead set assembly of claim 42 wherein said inline impedance controlling elements include at least one of a resistor, a capacitor and an inductor.

44. The lead set assembly of claim 26 wherein at least one of said conductive paths defines at least one gap therein across each of which an impedance controlling element is attached for enabling at least one of said condition sense lead and said noise pickup lead to be tuned to a desired impedance.

45. The lead set assembly of claim 26 further including:
(a) a first impedance load interconnecting said first and said second conductive paths of said condition sense lead; and
(b) a second impedance load interconnecting said third and said fourth conductive paths of said condition sense lead; said first and said second impedance loads enabling said condition sense lead to have an impedance greater than that existing at an interface between said electrodes and said subject.

46. The lead set assembly of claim 45 wherein said first and said second impedance loads each include at least one of a resistor, a capacitor and an inductor.

47. The lead set assembly of claim 26 further including:
(a) a third impedance load interconnecting said first and said second conductive paths of said noise pickup lead; and
(b) a fourth impedance load interconnecting said third and said fourth conductive paths of said noise pickup lead; said second and said third impedance loads enabling said noise pickup lead to have an impedance substantially equal to that existing at an interface between said electrodes and said subject.

48. The lead set assembly of claim 47 wherein said third and said fourth impedance loads each include at least one of a resistor, a capacitor and an inductor.

49. The lead set assembly of claim 26 further including a connector by which the lead set assembly, and said conductive paths thereof, can be coupled to said separate device.

50. A method of ascertaining a condition of a patient, the method comprising the steps of:
(a) removably associating a plurality of electrodes with the patient, each of said electrodes for picking up a bioelectric signal indicative of the condition of the patient;
(b) connecting a lead set assembly between said electrodes and a separate device, said lead set assembly including:
(I) a condition sense lead having a pair of conductive paths, each of said conductive paths being connectible to one of said electrodes for conveying a bioelectric signal therefrom and susceptible to having a first noise signal induced therein by electromagnetic emanations; and
(II) a noise pickup lead having a pair of conductive paths each of which being effectively topographically aligned with a corresponding one of said conductive paths of said condition sense lead to form a noise-matched pair therewith and susceptible to having a second noise signal induced therein by the electromagnetic emanations, thereby enabling said second noise signal to be substantially approximate to said first noise signal in each of said noise-matched pairs, said lead set assembly thus for conveying from said electrodes to said separate device (I) said bioelectric signals and said first noise signals using said conductive paths of said condition sense lead and (II) said second noise signals using said conductive paths of said noise pickup lead; and
(c) monitoring the condition of the patient through use of said bioelectric signals received by said separate device.

51. A magnetic resonance (MR) system comprising:
(a) a means for controlling gradient and radio frequency (RF) coils to apply changing magnetic fields and RF signals to a patient to induce and encode magnetic resonance within selected nuclei therein;
(b) a means for receiving magnetic resonance signals from the selected nuclei in response thereto and processing the magnetic resonance signals into diagnostic information;
(c) a plurality of electrodes removably attachable to the patient, each of said electrodes for picking up a bioelectric signal indicative of a condition of the patient; and
(d) a lead set assembly for conveying said bioelectric signals from said electrodes to a separate device, said lead set assembly comprising:
(I) a condition sense lead having a pair of conductive paths, each of said conductive paths being connectible to one of said electrodes for conveying a bioelectric signal therefrom and susceptible to having a first noise signal induced therein by electromagnetic emanations; and
(II) a noise pickup lead having a pair of conductive paths each of which being effectively topographically aligned with a corresponding one of said conductive paths of said condition sense lead to form a noise-matched pair therewith and susceptible to having a second noise signal induced therein by the electromagnetic emanations, thereby enabling said second noise signal to be substantially approximate to said first noise signal in each of said noise-matched pairs, said lead set assembly thus for conveying from said electrodes to said separate device (I) said bioelectric signals and said first noise signals using said conductive paths of said condition sense lead and (II) said second noise signals using said conductive paths of said noise pickup lead.
